(19) United States
(12) Patent Application Publication
(10) Pub. No.: US 2005/0245600 A1
(43) Pub. Date: Nov. 3, 2005

(54) Novel antimalarial agent

(75) Inventors: Yoshihiro Urade, Kyoto-shi (JP); Bruno Kilunga Kubata, Nairobi (KE); Nobutoshi Murakami, Osaka-fu (JP); Toshihiro Horii, Osaka-fu (JP)

Correspondence Address: BROWDY AND NEIMARK, P.L.L.C.;624 NINTH STREET, NW, SUITE 300, WASHINGTON, DC, 20001-5303, US

(73) Assignee: Osaka Bioscience Institute, Suita-shi (JP)

(21) Appl. No.: 10/518,053

(22) PCT Filed: Jun. 23, 2003
(86) PCT No.: PCT/JP03/07915

§ 371 Date: Dec. 16, 2004

(30) Foreign Application Priority Data
Jun. 24, 2002 (JP) 2002-182969

(51) Int. Cl. A61K 31/353

(52) U.S. Cl. 514/456

(57) ABSTRACT

A novel antimalarial agent. It is a medical composition comprising a pharmaceutically acceptable carrier and a compound represented by the general formula (I), wherein R₁ to R₁₂ is independently, hydrogen, halogen, hydroxy, alkyl, alkoxy, amino or acylamino.

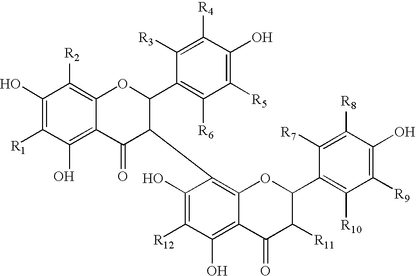

### TECHNICAL FIELD

The present invention relates to a novel antimalarial agent.

### BACKGROUND ART

Malaria is an ardent disease caused by four kinds of malaria parasite (Plasmodium) consisting of three days fever, four days fever, tropical fever and egg-type, and exhibits characteristic pyretic type. It has a life cycle in which human being and anopheles mosquito are host and mosquito mediates it. Among them, tropical fever type malaria parasite (Plasmodium falciparum) causes malignant malaria in the tropical area such as East and South Asia, South and West Africa and Middle America. There are two point two billions of resident in the malaria-raging region. The sufferers of malaria are about two hundreds and seventy millions for a year and the death toll from malaria is about two hundred millions for a year.

Furthermore, the following severe problems are pointed out (Nature, Vol. 415 (2002), p. 686):

1) Resistance to antimalarial agents: There are some antimalarial agents such as quinine, chloroquine, and sulphadoxine/pyrimethamine. However, malaria parasite having resistance to the antimalarial agents are increasing. In Southeast Asia, Plasmodium falciparum is now resistant to almost all antimalarial agents. In Africa, chloroquine resistance is widespread and resistance to sulphadoxine/pyrimethamine is increasingly detected; 2) Insecticide resistance: Mosquitoes resistant to pyrethroid insecticides in West and South Africa have emerged; 3) War: Wars in Africa and elsewhere have led to malaria transmission due to refugee people; 4) Climatic change: Global warming may have contributed to the spread of malaria into previously malaria-free area; 5) Travel: About 7,000 imported cases of malaria by travelers are recorded in Europe each year; 6) Population increase: During the past two decades, the population in many malaria-endemic area has doubled, thus greatly increasing absolute numbers of those at risk.

Accordingly, there is an increasing need for a novel antimalarial agent which is effective for treating the malaria resistant to conventional antimalarial agents.

### DISCLOSURE OF INVENTION

Technical Problem to be Solved

The present invention was made to solve the above-mentioned problems. That is, it is an object of the present invention to provide an antimalarial agent which is also effective for the treatment of malaria resistant to conventional antimalarial agent.

Solution for Problem

The present invention relates to a pharmaceutical composition used for the treatment of malaria comprising the compound of the general formula (I): 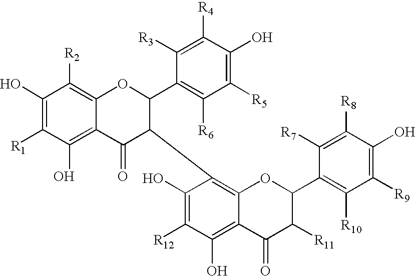
(wherein R₁ to R₁₂ are independently hydrogen atom, halogen atom, hydroxyl group, alkyl group, alkoxy group, or acylamino group)
and a pharmaceutically acceptable carrier.

The term “halogen atom” used herein means fluorine atom, chlorine atom, bromine atom and iodine atom.

The term “alkyl group” used herein involves a linear or branched alkyl group having 1 to 4 carbon atoms.

The term “alkoxy group” used herein means the above alkyl group bonded through oxygen atom.

The term “amino group” used herein involves, in addition to —NH₂ group, secondary or tertiary amino group in which one or two hydrogen atoms of —NH₂ group are substituted with the above mentioned alkyl group, respectively.

The term “acylamino group” used herein means RCONH— group wherein R is hydrogen atom or the above-mentioned alkyl group.

As apparent from the general formula (I), the compound used in the present invention has at least three asymmetric carbon atoms and there can be thus many stereoisomers. Not only individual stereoisomers but also the mixtures thereof are within the scope of the present invention.

### BEST MODE FOR CARRYING OUT THE INVENTION

According to one embodiment of the present invention, a compound in which R₁, R₂, R₃, R₄, R₅, R₆, R₇, R₈, R₉, R₁₀, R₁₁ and R₁₂ are hydrogen atom in the formula (I) can used as an antimalarial agent.

Among them, it is a preferred embodiment to use a compound represented by the formula: 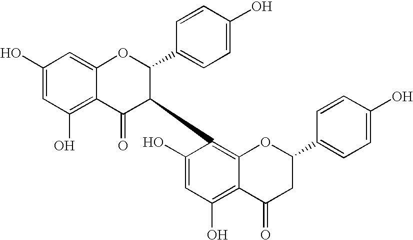
(2S,3R,2′S-5,7,5′,7′-tetrahydroxy-2,2′-bis-(4-hydroxyphenyl)-2,3,2′,3′-tetrahydro-[3,8′]bichromenyl-4,4′-dione) as an antimalarial agent.

According to another embodiment of the present invention, a compound in which R₁, R₂, R₃, R₄, R₅, R₆, R₇, R₈, R₉, R₁₀, and R₁₂ are hydrogen atom and R₁₁ is hydroxyl group in the formula (I) is used as an antimalarial agent.

Among them, it is a preferred embodiment to use a compound represented by the formula: 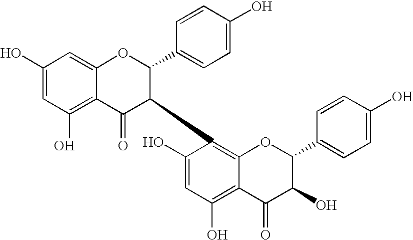
(2S,3R,2′S,3′R-5,7,3′,5′,7′-pentahydroxy-2,2′-bis-(4-hydroxyphenyl)-2,3,2′,3′-tetrahydro-[3,8′]bichromenyl-4,4′-dione) or a compound represented by the formula: 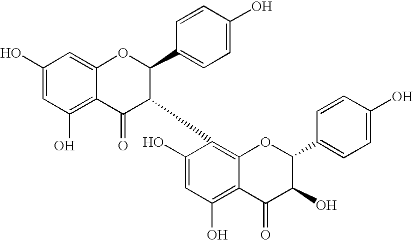
(2R,3S,2′S,3′R-5,7,3′,5′,7′-pentahydroxy-2,2′-bis-(4-hydroxyphenyl)-2,3,21,3′-tetrahydro-[3,8′]bichromenyl-4,4′-dione) as an antimalarial agent.

According to another embodiment of the present invention, a compound in which R₁, R₂, R₃, R₄, R₅, R₆, R₇, R₉, R₁₀, and R₁₂ are hydrogen atom and R₈ and R₁₁ are hydroxyl group is used as an antimalarial agent.

Among them, it is a preferred embodiment to use a compound represented by the formula: 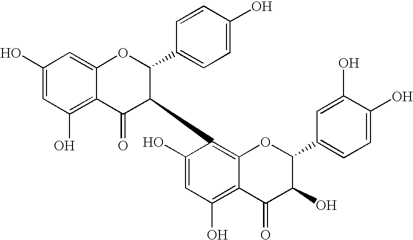
(2S,3R,2′S,3′R-2′-(3,4-dihydroxyphenyl)-5,7,3′,5′,7′-pentaahydroxy-2-(4-hydroxyphenyl)-2,3,2′,3′-tetrahydro-[3,8′]bichromenyl-4,4′-dione) as an antimalarial agent.

The compound represented by the general formula (I) used in the present composition can be isolated from plants such as Garcinia kola as exhibited in Example. It is also possible to prepare it by chemical synthesis.

### EXAMPLE 1

The compound of the present invention was extracted and isolated from Garcinia kola using anti-malarial activity as a guidance.

### (1) Extraction with 70% Ethanol

Four liters of 70% ethanol was added to 500 g of milled Garcinia kola seed and the mixture was subjected to extraction at room temperature for 24 hours. This procedure was further repeated twice. The mixture was then filtered and concentrated to give 27.12 g of solid (referred to as “EtOH extract” hereinafter).

### (2) CH₂Cl₂/H₂O Treatment

EtOH extract obtained in (1) was added to a 750 mL/800 mL mixture of CH₂Cl₂/H₂O and thoroughly stirred to distribute and dissolve it in both the layers. The same procedure was repeated for undissolved substance. The phase separation was effected and the CH₂Cl₂ layer was concentrated to dryness to give 7 g of a solid (referred to as “CH₂Cl₂ extract” hereinafter).).

### (3) Extraction with Ethyl Acetate

Seven hundreds and fifty milliliters of ethyl acetate were added to the aqueous layer obtained by the phase separation of (2). Seven hundreds and fifty-milliliters of ethyl acetate were further added to the aqueous layer and phase separated. The combined ethyl acetate layers were concentrated to dryness to give 10 g of a solid (referred to as “ethyl acetate extract” hereinafter)

### (4) Extraction with n-Butanol

To the aqueous layer obtained by the phase separation of (3), 500 mL of butanol was added. The mixture was thoroughly stirred and subjected to phase separation. To the aqueous solution, 500 mL of butanol was further added and subjected to phase separation. The combined butanol layers were concentrated to dryness to give 6 g of a solid (referred to as “n-butanol extract” hereinafter). The aqueous layers were concentrated to dryness to give 3 g of a solid (referred to as “water extract” hereinafter).

### (5) Measurement of Anti-Plasmodium falciparum Activity of Extracts

Plasmodium falciparum growth inhibition activity of each extracts obtained in (1) to (4) was determined as follows.

FCR3 strain (resistant to cycloguanyl) of Plasmodium falciparum isolated in Gambia was used in the experiment to assess anti-malarial activity. Plasmodium falciparum synchronized to a ring phase by sorbitol treatment was injected in 50 μL 96 well plate. The hematocrit value of the culture liquid is 2% and the infection ratio was 0.55%. A substance to be tested was dissolved in DMSO and diluted with medium to an appropriate concentration before 50 μL of a test compound solution was added to the above 96 well plate to the total volume of 100 μL. The final concentration of DMSO added was 1%. Subsequently, after incubation was effected at 37° C. for 48 hours, a sumea film prepared on a slide glass was stained with Giemsa solution. The number of infected erythrocyte among 1000 erythrocytes was counted under an optical microscope. Percent inhibition of malaria parasite growth was calculated for percent infection obtained under the presence of DMSO only and that under the presence of the compound to be tested. Quinine was used as a positive control. The results are shown in Table 1.

TABLE 1
P. falciparum Growth Inhibition (%)

Concentration

| Sample | 5 μg/mL | 0.5 μg/mL |
|---|---|---|
| EtOH extract | 87% | 73% |
| CH₂Cl₂ extract | 88 | 77 |
| Ethyl acetate extract | 90 | 72 |
| n-butanol extract | 90 | 82 |
| water extract | 0 | 0 |

control: quinine (100 ng/mL) 90%; (33 ng/mL) 73%

### (6) Fractionation by Silica Gel Chromatography

Three grams of the combined sample of CH₂Cl₂ extract obtained in (2) and ethyl acetate extract obtained in (3) were fractionated using silica gel chromatography. Three grams of the sample were adsorbed on glass column (filling solvent CHCl₃:EtOAC=90:10) which had been packed with SiO₂ (80 g) and an eluting solution (CHCl₃:EtOAC=90:10) was passed through the column to give Fraction A (249 mg). Furthermore, the change of the eluting solvent gave the following fractions: Fraction B (537 mg, eluting solution CHCl₃:EtOAC=70:30); Fraction C (82 mg, eluting solution CHCl₃:EtOAC=60:40); Fraction D (445 mg, eluting solution CHCl₃:EtOAC=50:50);

Fraction E (655 mg, eluting solution CHCl₃:EtOAC=40:60);

Fraction F (325 mg, eluting solution EtOAC);

Fraction G (340 mg, eluting solution MeOH).

Fractions A (249 mg), B (537 mg), C (82 mg), D (445 mg), E (655 mg), and F (325 mg)were thus obtained.

### (7) Antimalarial Activity of Fractions A to G

Antimalarial activity of fraction A to G was measured in the similar way with (5). Percent growth inhibition of human cancer cell KB3-1 was also measured to examine the toxicity of Fractions A to G as follows:

To 100 μL (2×10⁴/ml) of floating liquid of KB3-1 cells, 100 μL of test compound containing a liquid which was prepared by dissolving a test compound in DMSO and diluted to an appropriate concentration (final concentration of DMSO: 1%) was added. The mixture was incubated under 5% CO₂ at 37° C. After 72 hours incubation, 25 μL of MTT reagent was added and incubation was effected for 3 hours before only medium was aspiratedly removed. To the medium 200 μL of DMSO was added, the resulting MTT folmazan was extracted and the amount of the dye was quantitatively measured by a calorimetric method (540 nm). The number of survival cell was counted from the amount of formed dye, and percent growth inhibition was determined to assess the toxic activity. Mitomycin C was used as a positive control. The results are given in Table 2.

TABLE 2
Antimalarial Activity of Fractions A to G

| | Inhibition of | | Inhibition of | |
|---|---|---|---|---|
| | P. falciparum %¹⁾ | | KB3-1 %²⁾ | |
| | 5 μg/mL | 0.5 μg/mL | 50 μg/mL | 5 μg/mL |
| Fraction A | 83% | 79% | 80% | 8% |
| Fraction B | 77 | 56 | 88 | 17 |
| Fraction C | 60 | 65 | 80 | 5 |
| Fraction D | 60 | 68 | 40 | 4 |
| Fraction E | 65 | 70 | 10 | 22 |
| Fraction F | 82 | 70 | 0 | 6 |
| Fraction G | 78 | 75 | 20 | 4 |
| EtOAc + CH₂Cl₂ | 84 | 72 | 11 | 5 |
| extract | | | | |

¹⁾control: quinine 0.1 μg/mL 90%; 0.033 μg/mL 62%
²⁾control: mytomycin C 1 μg/mL 43%

### (8) Fractionation of Fraction D with ODS Column

Fraction D obtained above (445 mg) was subjected to fractionation with ODS column as follows:

Four hundreds forty five milligrams of the sample were adsorbed on glass column (filling solvent MeOH: H₂O=45:55) packed with ODS (15 g) and an eluting solution (MeOH: H₂O=45:55) passed through the column to give Fraction D1 (280 mg) and Fraction D2 (41 mg). In addition, eluting solvent is changed to MeOH to give Fraction D3 (80 mg). Fractions D1 (280 mg), D2 (41 mg), and D3 (80 mg) were thus obtained.

Fraction D2 was fractionated with ODS HPLC as follows:

Fraction D2 (41 mg) was fractionated using a column (cosmosil C18 10 mm i.d.×250 mm) under the condition of mobile phase (CH₃CN:H₂O:TFA=40:60:0.1) at a flow rate of 3.0 mL/min with UV detection at 240 nm to give Fraction GK-1(33 mg). Fraction D1 was a mixture of GK-1 and GK-2 in 1:1.

### (9) Fractionation of Fraction F with ODS HPLC

Fraction F (100 mg) was subjected to fractionation with ODS HPLC as follows: Fraction F (100 mg) was fractionated using a column (cosmosil C18 10 mm i.d.×250 mm) under the condition of mobile phase (MeOH:H₂O=50:50) at a flow rate of 2.5 mL/min with UV detection at 240 nm to give Fraction GK-3 (24 mg) and GK-4 (36 mg).

### (10) Identification of Each Fraction

Fractions GK-1, E(referred to as “GK-2” hereinafter), GK-3 and GK-4 were measured with 1H NMR, ¹³C NMR, and FAB-MS to identify compounds.

### (10-1) GK-1

¹H NMR (500 MHz, DMSO-d₆) δ: 12.17 (1H, s, II-OH-5), 12.01 (1H, s, II-OH-5), 10.82 (1H, s, I-OH-7), 10.73 (1H, s, II-OH-7), 9.45 (2H, s, I-OH-4′, II-OH-4′), 7.20 (2H, d, J=8.0 Hz, II-2′, 6′), 7.10 (2H, d, J=8.0 Hz, I-2′, 6′), 6.64 (2H, d, J=8.0 Hz, I-3′, 5′), 6.62 (2H, d, J=8.0 Hz, II-3′, 5′), 5.90 (1H, s, I-6), 5.84 (1H, s, I-8), 5.79 (1H, s, II-6), 5.56 (1H, d, J=12.2 Hz, I-2), 5.38 (1H, d, J=12.2 Hz, II-2), 4.66 (H, d, J=12.2 Hz, I-3), 2.94 (1H, m, II-3a), 2.74 (1H, m, II-3b). ¹³C NMR (125 MHz, DMSO-d₆) δc: 196.4 (II-4), 195.9 (I-4), 166.3 (I-7), 164.3 (II-7), 163.5 (I-5), 162.5 (II-5), 161.9 (I-9), 159.6 (II-9), 157.4 (I-4′), 157.0 (II-4′), 129.0 (II-1′), 128.8 (I-2′, 6′), 127.9 (I-1′), 126.5 (II-2′, 6′), 114.8 (II-3′, 5′), 114.6 (I-3′, 5′), 101.4 (II-8), 101.3 (II-10), 101.2 (I-10), 96.0 (I-6), 95.0 (I-8), 94.9 (II-6), 81.2 (I-2), 78.1 (II-2), 47.3 (I-3), 42.6 (II-3). ¹H NMR (500 MHz, DMSO-d₆) atropisomer δ: 12.28 (1H, s, I-OH-5), 12.13 (1H, s, II-OH-5), 11.17 (1H, s, I-OH-7), 10.73 (1H, s, II-OH-7), 9.59 (1H, s, II-OH-4′), 9.52 (1H, s, I-OH-4′), 7.13 (2H, d, J=8.0 Hz, II-2′, 6′), 7.10 (2H, d, J=8.0 Hz, I-2′, 6′), 6.83 (2H, d, J=8.0 Hz, II-3′, 5′), 6.70 (2H, d, J=8.0 Hz, I-3′, 5′), 5.94 (1H, s, II-6), 5.90 (1H, s, I-6), 5.76 (1H, s, I-8), 5.68 (1H, d, J=12.2 Hz, I-2), 5.38 (1H, d, J=12.2 Hz, II-2), 4.51 (H, d, J=12.2 Hz, I-3), 2.74 (1H, m, II-3a), 2.58 (1H, m, II-3b). ¹³C NMR (125 MHz, DMSO-d₆) atropisomer δc: 196.6 (II-4), 195.9 (I-4), 166.3 (I-7), 164.8 (II-7), 163.7 (I-5), 162.6 (II-5), 162.3 (I-9), 160.6 (II-9), 157.6 (I-4′, II-4′), 129.3 (II-11′), 128.8 (I-2′, 6′), 127.9 (I-1′), 127.6 (II-2′, 6′), 115.1 (II-3′, 5′), 114.6 (I-3′, 5′), 101.8 (I-10), 101.4 (II-8), 101.3 (II-10), 96.0 (I-6), 95.5 (I-8), 94.9 (II-6), 81.6 (I-2), 78.3 (II-2), 47.3 (I-3), 43.0 (II-3). FAB-MS: 543 (M+H)+

From these data, GK-1 is considered to be the compound (2S,3R,2′S-5,7,5′,7′-tetrahydroxy-2,2′-bis-(4-hydroxyphenyl)-2,3,2′,3′-tetrahydoro-[3,8′]bichromenyl-4,4′-dione) having the following formula:

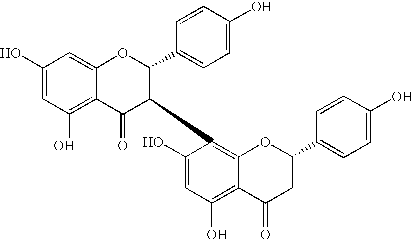

### (10-2) GK-2

¹H NMR (500 MHz, DMSO-d₆) δ: 12.27 (1H, s, I-OH-5), 11.83 (1H, s, II-OH-5), 11.18 (1H, s, I-OH-7), 10.69 (1H, s, II-OH-7), 9.53 (1H, s, II-OH-4′), 9.51 (1H, s, I-OH-4′), 7.16 (2H, d, J=8.0 Hz, II-2′, 6′), 7.07 (2H, d, J=8.0 Hz, I-2′, 6′), 6.82 (2H, d, J=8.0 Hz, II-3′, 5′), 6.74 (2H, d, J=8.0 Hz, I-3′, 5′), 5.93 (1H, s, II-6), 5.88 (1H, s, I-6), 5.75 (1H, s, I-8), 5.72 (1H, d, J=5.5 Hz, II-OH-3), 5.64 (1H, d, J=12.2 Hz, I-2), 4.96 (1H, d, J=12.2 Hz, II-2), 4.42 (H, d, J=12.2 Hz, I-3), 3.98 (1H, dd, J=12.2, 5.5 Hz, II-3); ¹³C NMR (125 MHz, DMSO-d₆) δc: 197.4 (II-4), 196.3 (I-4), 166.2 (I-7), 164.4 (II-7), 163.7 (I-5), 162.8 (II-5), 162.5 (I-9), 160.8 (II-9), 157.6 (I-4′, II-4′)), 128.9 (II-2′, 6′), 128.6 (I-2′, 6′), 128.1 (I-1′), 127.4 (II-1′), 114.8 (II-3′, 5′), 114.6 (I-3′, 5′), 101.2 (I-10), 101.1 (II-8), 99.7 (II-10), 96.0 (I-6), 95.7 (II-6), 94.9 (I-8), 82.4 (II-2), 81.1 (I-2), 71.8 (II-3), 47.3 (I-3). ¹H NMR (500 MHz, DMSO-d₆) atropisomer δ: 12.14 (1H, s, I-OH-5), 11.70 (1H, s, II-OH-5), 10.82 (1H, s, I-OH-7), 10.65 (1H, s, II-OH-7), 9.42 (1H, s, I-OH-4′), 9.38 (1H, s, II-OH-4′), 7.16 (2H, d, J=8.0 Hz, II-2′, 6′), 7.07 (2H, d, J=8.0 Hz, I-2′, 6′), 6.64 (4H, d, J=8.0 Hz, I-3′, 5′, II-3′, 5′), 5.88 (1H, s, I-6), 5.85 (1H, s, I-8), 5.83 (1H, d, J=5.5 Hz, II-OH-3), 5.79 (1H, s, II-6), 5.31 (1H, d, J=12.2 Hz, I-2), 5.13 (1H, d, J=12.2 Hz, II-2), 4.64 (1H, d, J=12.2 Hz, I-3), 4.22 (1H, dd, J=12.2, 5.5 Hz, II-3). ¹³C NMR (125 MHz, DMSO-d₆) atropisomerbc: 197.4 (II-4), 196.6 (I-4), 166.3 (I-7), 164.9 (II-7), 163.5 (I-5), 162.5 (I-9), 161.7 (II-5), 159.3 (II-9), 157.5 (I-4′), 157.2 (II-4′), 128.8 (I-2′, 6′), 128.1 (II-2′, 6′), 127.8 (II-11′), 127.7 (I-1′), 114.6 (I-3′, 5′, II-3′, 5′), 101.2 (I-10), 101.1 (II-8), 100.2 (II-10), 96.0 (I-6), 95.3 (II-6), 94.9 (I-8), 82.4 (II-2), 81.6 (I-2), 72.3 (II-3), 47.3 (I-3). FAB-MS: 559 (M+H)+

From these data, GK-2 is considered to be the compound (2S,3R,2′S,3′R-5,7,3′,5′,7′-pentahydroxy-2,2′-bis-(4-hydroxyphenyl)-2,3,2′,3′-tetrahydro-[3,8′]bichromenyl-4,4′-dione) having the following formula:

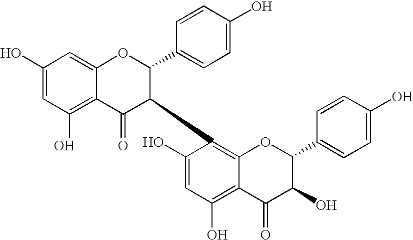

### (10-3) GK-3

¹H NMR (500 MHz, DMSO-d₆) δ: 12.19 (1H, s, I-OH-5), 11.83 (1H, s, II-OH-5), 10.76 (1H, s, II-OH-7), 9.55 (1H, s, I-OH-4′), 8.95 (1H, s, II-OH-4′), 8.92 (1H, s, II-OH-3′), 7.09 (2H, m, I-2′, 6′), 6.83 (1H, s, II-2′), 6.79 (1H, d, J=8.0 Hz, II-6′), 6.76 (1H, d, J=8.0 Hz, II-5′), 6.64 (2H, d, J=8.0 Hz, I-3′, 5′), 5.91 (1H, s, II-6), 5.87 (1H, s, I-6), 5.75 (1H, s, I-8), 5.72 (1H, d, J=5.5 Hz, II-OH-3), 5.67 (1H, d, J=12.2 Hz, I-2), 4.88 (1H, d, J=12.2 Hz, II-2), 4.46 (1H, d, J=12.2 Hz, I-3), 3.96 (1H, dd, J=12.2, 5.5 Hz, II-3); ¹³C NMR (125 MHz, DMSO-d₆) δc: 197.4 (II-4), 196.2 (I-4), 166.2 (I-7), 164.3 (II-7), 163.4 (I-5), 162.4 (I-9), 162.0 (II-5), 160.0 (II-9), 157.9 (II-4′), 145.2 (II-4′), 144.4 (II-3′), 128.7 (I-2′, 6′), 127.7 (I-1′, II-1′), 117.2 (II-6′), 115.2 (II-5′), 114.6 (I-3′, 5′), 101.2 (I-10), 101.1 (II-8), 99.6 (II-10), 95.9 (I-6), 95.6 (II-6), 94.8 (I-8), 82.6 (II-2), 81.1 (I-2), 71.7 (II-3), 47.0 (I-3). ¹H NMR (500 MHz, DMSO-d₆) atropisomer δ: 12.12 (1H, s, I-OH-5), 11.76 (1H, s, II-OH-5), 10.71 (1H, s, II-OH-7), 9.49 (1H, s, I-OH-4′), 9.11 (1H, s, II-OH-4′), 8.83 (1H, s, II-OH-3′), 7.09 (2H, m, I-2′, 6′), 6.76 (2H, m, II-2′, 6′), 6.64 (2H, d, J=8.0 Hz, I-3′, 5′), 6.59 (1H, d, J=8.0 Hz, II-5′), 5.87 (1H, s, I-6), 5.85 (1H, d, J=5.5 Hz, II-OH-3), 5.84 (1H, s, I-8), 5.80 (1H, s, II-6), 5.35 (1H, d, J=12.2 Hz, I-2), 5.00 (1H, d, J=12.2 Hz, II-2), 4.66 (1H, d, J=12.2 Hz, I-3), 4.19 (1H, dd, J=12.2, 5.5 Hz, II-3). 13C NMR (125 MHz, DMSO-d6) atropisomer δc: 197.4 (II-4), 196.2 (I-4), 166.2 (I-7), 164.8 (II-7), 163.4 (I-5), 162.6 (I-9), 161.6 (II-5), 159.3 (II-9), 157.4 (I-4′), 145.7 (II-4′), 144.8 (II-3′), 128.7 (I-2′, 6′), 127.9 (I-1′, II-1′), 118.8 (II-6′), 115.2. (II-5′), 114.6 (I-3′, 5′), 101.2 (II-10), 101.1 (I-10), 100.1 (II-8), 95.9 (I-6), 95.2 (II-6), 94.8 (I-8), 82.6 (II-2), 81.5 (I-2), 72.1 (II-3), 47.0 (I-3). FAB-MS: 575 (M+H)+

From these data, GK-3 is considered to be the compound (2S,3R,2′S,3′R-2′-(3,4-dihydroxyphenyl)-5,7,3′,5′,7′-pentahydroxy-2-(4-hydroxyphenyl)-2,3,2′,3′-tetrahydro-[3,8′]bichromenyl-4,4′-dione) having the following formula:

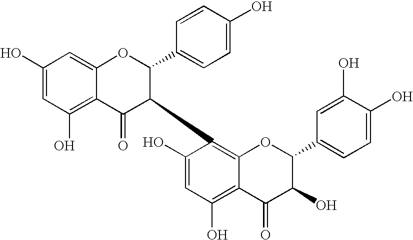

### (10-4) GK-4

¹H NMR (500 MHz, DMSO-d₆) δ: 12.18 (1H, s, I-OH-5), 11.73 (1H, s, II-OH-5), 9.53 (1H, s, I-OH-4′), 9.38 (1H, s, II-OH-4′), 7.16 (2H, d, J=8.0 Hz, II-2′, 6′), 7.08 (2H, d, J=8.0 Hz, I-2′, 6′), 6.64 (4H, d, J=8.0 Hz, I-3′, 5′, II-3′, 5′), 5.88 (1H, s, I-6), 5.85 (1H, d, J=5.5 Hz, II-OH-3), 5.84 (1H, s, I-8), 5.78 (1H, s, II-6), 5.32 (1H, d, J=12.2 Hz, I-2), 5.12 (1H, d, J=12.2 Hz, II-2), 4.66 (1H, d, J=12.2 Hz, I-3), 4.21 (1H, br.d, J=12.2 Hz, II-3). ¹³C NMR (125 MHz, DMSO-d₆) δc: 197.1 (II-4), 196.8 (I-4), 166.4 (I-7), 165.5 (II-7), 163.7 (I-5), 162.6 (I-9), 161.7 (II-5), 159.3 (II-9), 157.5 (I-4′), 157.2 (II-4′), 128.9 (I-2′, 6′), 128.0 (II-2′, 6′), 127.8 (II-1′), 127.7 (I-1′), 114.6 (I-3′, 5′, II-3′, 5′), 101.3 (I-10, II-8, 10), 96.0 (I-6), 95.4 (II-6), 94.9 (I-8), 82.4 (II-2), 81.7(I-2), 72.7 (II-3), 47.2 (I-3). ¹H NMR (500 MHz, DMSO-d₆) atropisomer δ: 12.28 (1H, s, I-OH-5), 11.86 (1H, s, II-OH-5), 9.53 (2H, s, I-OH-4′, II-OH-4′), 7.16 (2H, d, J=8.0 Hz, II-2′, 6′), 7.08 (2H, d, J=8.0 Hz, I-2′, 6′), 6.83 (2H, d, J=8.0 Hz, II-3′, 5′), 6.74 (2H, d, J=8.0 Hz, I-3′, 5′), 5.89 (1H, s, II-6), 5.88 (1H, s, I-6), 5.75 (1H, s, I-8), 5.72 (1H, d, J=5.5 Hz, II-OH-3), 5.66 (1H, d, J=12.2 Hz, I-2), 4.94 (1H, d, J=12.2 Hz, II-2), 4.39 (H, d, J=12.2 Hz, I-3), 3.96 (1H, dd, J=12.2, 5.5 Hz, II-3). ¹³C NMR (125 MHz, DMSO-d₆) atropisomer δc: 197.8 (II-4), 196.4 (I-4), 166.3 (I-7), 163.7 (II-7), 162.5 (I-9), 162.1 (II-5), 160.0 (II-9), 159.4 (I-5), 157.7 (II-4′), 157.6 (I-4′), 129.0 (II-2′, 6′), 128.2 (I-1′), 128.0 (I-2′, 6′), 127.5 (II-1′), 114.8 (II-3′, 5′), 114.6 (I-3′, 5′), 101.3 (II-8), 100.0 (I-10), 99.3 (II-10), 96.0 (I-6, II-6), 94.9 (I-8), 82.4 (II-2), 81.1 (I-2), 71.8 (II-3), 47.2 (I-3). FAB-MS: 559 (M+H)+

From these data, GK-4 is considered to be the compound (2R,3S,2′S,3′R-5,7,3′,5′,7′-pentahydroxy-2,2′-bis-(4-hydroxyphenyl)-2,3,2′,3′-tetrahydro-[3,8′]bichromenyl-4,4′-dione) having the following formula:

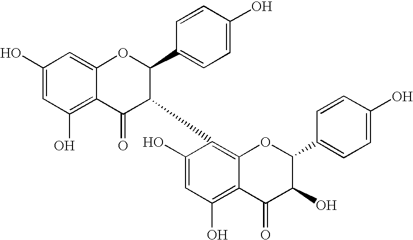

### EXAMPLE 2

In Vitro Antimalarial Activity Assay

Antimalarial activity and toxicity of the above identified compounds were assayed. Antimalarial activity assay was carried out as described in (5) and toxicity assay was carried out as described in (7). The results are indicated as IC₅₀ and the ratio thereof is shown as an index of selective toxicity

TABLE 3
Antimalarial activity and selective toxicity of each compound

| | P. falciparum¹⁾ | KB3-1²⁾ | Selective |
|---|---|---|---|
| compound | (IC₅₀, μg/mL) | (IC₅₀, μg/mL) | toxicity³⁾ |
| GK-1 | 0.35 | 27 | 77 |
| GK-2 | 0.09 | >100 | >1100 |
| GK-3 | 0.12 | >100 | >830 |
| GK-4 | 1.1 | >100 | >91 |

¹⁾Control P. falciparum quinine 0.1 μg/mL: 82%; 0.033 μg/mL: 36%
²⁾Control KB3-1 mytomycineC 1 μg/mL: 39%
³⁾IC₅₀ (KB3-1)/IC₅₀ (P. falciparum)

### EXAMPLE 3

### In Vivo Antimalarial Activity Assay

For GK-2 which exhibited the strongest action in the in vitro antimalarial activity assay, in vivo antimalarial activity was examined by four days inhibition effect using Plasmodium berghei (NK65 line) infected mouse(5 weeks old male ICR mouser body weight 22-25 g).

Four weeks old male mouse was bred at 25° C. under the condition of 12 hours light and 12 hours dark, and used in the assay.

Blood was corrected with a syringe from the heart of a donor mouse with 15 % of parasitaemia. To this blood, trisodium citrate aqueous solution was added in one seventh by volume to prevent coagulation of blood. The resulting solution was diluted with 0.9% aqueous sodium chloride solution so that the final concentration of infected blood cells is 1×10⁷ per 0.2 ml. Test compound was suspended with 0.5% CMC aqueous solution so that the concentration of the test compound was 25, 50, 100, and 200 mg/kg, respectively.

To mouse (one group: 5), 0.2 ml of the infected erythrocytes was administered through tail vein. After 2 hours, the CMC solution (0.2 ml) of the respective concentration prepared above was orally administered. Starting with this time as 0 day, the solution was administered every day till a third day and infection ratio was examined on a fourth day. The infection ratio was determined by collecting blood from mouse tail, preparing smear, and counting infected erythrocytes using a microscope. The infection ratio of control mouse (5 infected mice administered only by the 0.5% CMC solution) was about 28% on the fourth day.

Percent growth inhibition of parasites by test compound was calculated from the following formula:
Percent growth inhibition of parasites=(1−averaged infection ratio of mice treated with test compound/averaged infection ratio of control mice)×100

Life prolonging effect by test compound (T/C) was calculated from the following equation.
Life Prolonging Effect (T/C)=averaged number of subsistence days of mouse treated by test compound/averaged number of subsistence days of control mouse

These results are given in Table 4.

TABLE 4
In Vivo Activity of GK-2 by Oral Administration

| | | Averaged | | Averaged | |
|---|---|---|---|---|---|
| | Dose | parasite blood | Inhibition | subsistence | |
| Treatment | (mg/kg) | (%)^{a} | (%) | days | T/C |
| GK-2 | 25 | 17.3 ± 3.4 | 40 | 9.5 ± 1.7* | 119 |
| | 50 | 16.6 ± 2.4 | 43 | 10.0 ± 0.0* | 125 |
| | 100 | 13.9 ± 2.4 | 52 | 10.0 ± 0.0* | 125 |
| | 200 | 11.5 ± 1.2 | 60 | 11.0 ± 0.7* | 134 |
| artemisinin | 15 | 13.3 ± 1.6 | 54 | 10.3 ± 2.3* | 129 |
| Control^{b} | | 28.9 ± 2.3 | 0 | 8.0 ± 0.0 | 100 |

^{a}Average ± SD calculated from 5 mices of each group
^{b}treated with 0.5% CMC (0.2 mL)
*statistically significant from control (p < 0.01, F-test)

Formulation

As apparent from the tables, the compound of the present invention has high antimalarial function and low toxity.

The compound of the present invention can be used in various dosage forms for administration purpose based on its pharmacological action. The pharmaceutical composition of the present invention can be prepared by homogeneously admixing the compound of the general formula (I) as an active ingredient with a pharmaceutically acceptable carrier. The carrier can be of wide form depending on the desired form of formulation for administration. The pharmaceutical composition has preferably a unit dosage form for the oral or injection administration. Any useful pharmaceutically acceptable carrier can be used for the composition in the form of oral administration. For example, oral liquid preparation such as suspension, and syrup can be prepared using water, saccharides such as sucrose, sorbitol, fructose; glycols such as polyethylene glycol and propylene glycol, oils such as sesame oil, sweet oil, soybean oil, preservative such as alkylparaban benzoate, flavor such as strawberry flavor and peppermint.

Powder, pill, capsule and tablet can be prepared using excipient such as lactose, glucose, sucrose and mannitol; disintegrant such as starch and sodium alginate, lubricant such as magnesium stearate and talc, binding agent such as polyvinyl alcohol, hydroxypropyl cellulose, and gelatin, surfactant such as fatty acid ester, and plastisizer such as glycerin. Tablet and capsule are the most useful unit oral dosage formulation due to easy administration. For preparation of tablet and capsule, solid carrier can be used. Solution for injection can be prepared using carrier consisting of aqueous solution.

Effective Amount of Administration

The medicament of the present invention is administered orally or by injection and the effective amount of administration is 1 to 100 mg/kg/day, preferably 10 to 50 mg/kg/day. Administration frequency is preferably about three times a day.

Formulation 1
Gelatin hard capsule of the following composition was
prepared by conventional procedure.

| | |
|---|---|
| Active ingredient | 25 mg |
| Starch | 150 mg |
| Magnesium stearate | 10 mg |

Formulation 2
Tablet of the following composition was
prepared by conventional procedure.

| | |
|---|---|
| Active ingredient | 25 mg |
| Cellulose, microcrystalline | 275 mg |
| SiO₂ | 10 mg |
| Magnesium stearate | 5 mg |

1. A pharmaceutical composition used for the treatment of malaria comprising the compound of the general formula: 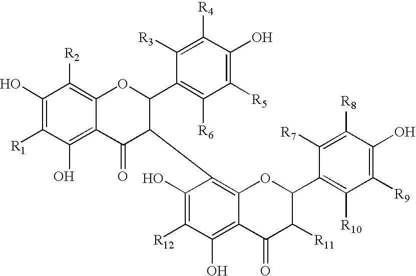
(wherein R₁˜R₁₂ is independently hydrogen atom, halogen atom, hydroxyl group, alkyl group, alkoxy group, amino group or acylamino group)
and a pharmaceutically acceptable carrier:

2. A pharmaceutical composition of claim 1 wherein R₁, R₂, R₃, R₄, R₅, R₆, R₇, R₈, R₉, R₁₀, R₁₁, and R₁₂ is hydrogen atom in the compound.

3. A pharmaceutical composition of claim 2 wherein the compound is 2S, 3R, 2′S-5,7,5′,7′-tetrahydroxy-2,2′-bis-(4-hydroxyphenyl)-2,3,2′,3′-tetrahydro-[3,8′]bichromenyl-4,4′-dione.

4. A pharmaceutical composition of claim 1 wherein R₁, R₂, R₃, R₄, R₅, R₆, R₇, R₈, R₉, R₁₀, and R₁₂ is hydrogen atom and R₁₁ is hydroxyl group in the compound.

5. A pharmaceutical composition of claim 4 wherein the compound is 2S,3R,2′S,3′R-5,7,3′,5′,7′-pentahydroxy-2,2′-bis-(4-hydroxyphenyl)-2,3,2′,3′-tetrahydro-[3,8′]bichromenyl-4,4′-dione.

6. A pharmaceutical composition of claim 4 wherein the compound is 2R,3S,2′S,3′R-5,7,3′,5′,7′-pentahydroxy-2,2′-bis-(4-hydroxyphenyl)-2,3,2′,3′-tetrahydro-[3,8′]bichromenyl-4,4′-dione.

7. A pharmaceutical composition of claim 1 wherein R₁, R₂, R₃, R₄, R₅, R₆, R₇, R₉, R₁₀, and R₁₂ is hydrogen atom and R₈ and R₁₁ is hydroxyl group.

8. A pharmaceutical composition of claim 7 wherein the compound is 2S,3R,2′S,3′R-2′-(3,4-dihydroxyphenyl)-5,7,3′,5′,7′-pentahydroxy-2-(4-hydroxyphenyl)-2,3,2′,3′-tetrahydro-[3,8′]bichromenyl-4,4′-dione.
